(19) United States
(12) Patent Application Publication
(10) Pub. No.: US 2008/0160557 A1
(43) Pub. Date: Jul. 3, 2008

(54) Diagnostic Test for Head and Facial Pain

(76) Inventor: Roger K. Cady, Ozark, MO (US)

Correspondence Address: HUSCH BLACKWELL SANDERS LLP, 190 CARONDELET PLAZA, SUITE 600, ST. LOUIS, MO, 63105-3441, US

(21) Appl. No.: 11/863,369

(22) Filed: Sep. 28, 2007

Related U.S. Application Data
(60) Provisional application No. 60/827,340, filed on Sep. 28, 2006.

(51) Int. Cl. C12Q 1/40; G01N 33/543; G01N 33/566

(52) U.S. Cl. 435/22; 436/518; 436/501

(57) ABSTRACT

A diagnostic test for head and facial pain includes providing a series of antibodies to several biological markers on a test strip, collecting a sample of saliva, and applying the saliva sample to the series of antibodies. If one or more of the anticipated biological markers are present in the sample of saliva, those markers bind to their correlating antibodies thereby providing an indicator of the presence and amount of a particular biological marker in the patient's saliva upon evaluation of the test strip. When applied to a suitable biosensor, the described method can generate an electrical signal that can subsequently be transmitted to other devices. The biosensor includes a substrate that is coated with D-poly lysine on which the various antibodies are randomly arranged. The interaction of biological marker and antibody alters the electrical impedance of the substrate.

### CROSS REFERENCE

This application claims the priority of provisional application Ser. No. 60/827,340, filed Sep. 28, 2006.

### BACKGROUND OF THE INVENTION

The present invention relates generally to the health field and more particularly to a diagnostic testing method for head and facial pain.

### BACKGROUND OF THE INVENTION

Facial pain disorders are common and cause substantial disability and work absenteeism. For example, migraine affects an estimated 12% of the adult population and is estimated to account for over 150 million days of work absenteeism. Beyond migraine, other common head and facial pain disorders include tension headache, migrainous headache, cluster headache, and sinusitis and tempromandibular joint dysfunction. Also, asthma and rhinosinusitis are other common and disabling medical conditions.

The differentiation of these disorders is largely based on clinical symptomatology and as such is a common cause of misdiagnosis which leads to in appropriate treatment. In addition there is a medical need to gauge the severity and progression of these diseases over time.

The present invention is directed to overcoming one or more of the problems set forth above.

### SUMMARY OF THE INVENTION

One aspect of the invention generally pertains to a method for rapid and non-invasive diagnostic testing for specific biological markers relevant to the diagnosis and monitoring of headache and facial pain.

Another aspect of the invention is to provide a method for a diagnostic testing method for headache and facial pain that results in a transmittable electrical signal correlating to an identified biological marker.

In one embodiment of the invention, there is provided a diagnostic test for head and facial pain that includes providing a series of antibodies to several biological markers on a test strip, collecting a sample of saliva, and applying the saliva sample to the series of antibodies. If one or more of the anticipated biological markers are present in the sample of saliva, those markers bind to their correlating antibodies thereby providing an indicator of the presence and amount of a particular biological marker in the patient's saliva upon evaluation of the test strip.

In another embodiment of the invention, the described method is applied to a suitable biosensor to generate an electrical signal that can subsequently be transmitted to other devices. The biosensor includes a substrate that is coated with D-poly lysine on which the various antibodies are randomly arranged. The interaction of biological marker and antibody alters the electrical impedance of the substrate.

These aspects are merely illustrative of the innumerable aspects associated with the present invention and should not be deemed as limiting in any manner. These and other aspects, features and advantages of the present invention will become apparent from the following detailed description when taken in conjunction with the referenced drawings.

### BRIEF DESCRIPTION OF THE DRAWINGS

Reference is now made more particularly to the drawings, which illustrate the best presently known mode of carrying out the invention and wherein similar reference characters indicate the same parts throughout the views.

FIG. 1 is a schematic diagram of a diagnostic test for head and facial pain according to one embodiment of the present invention.

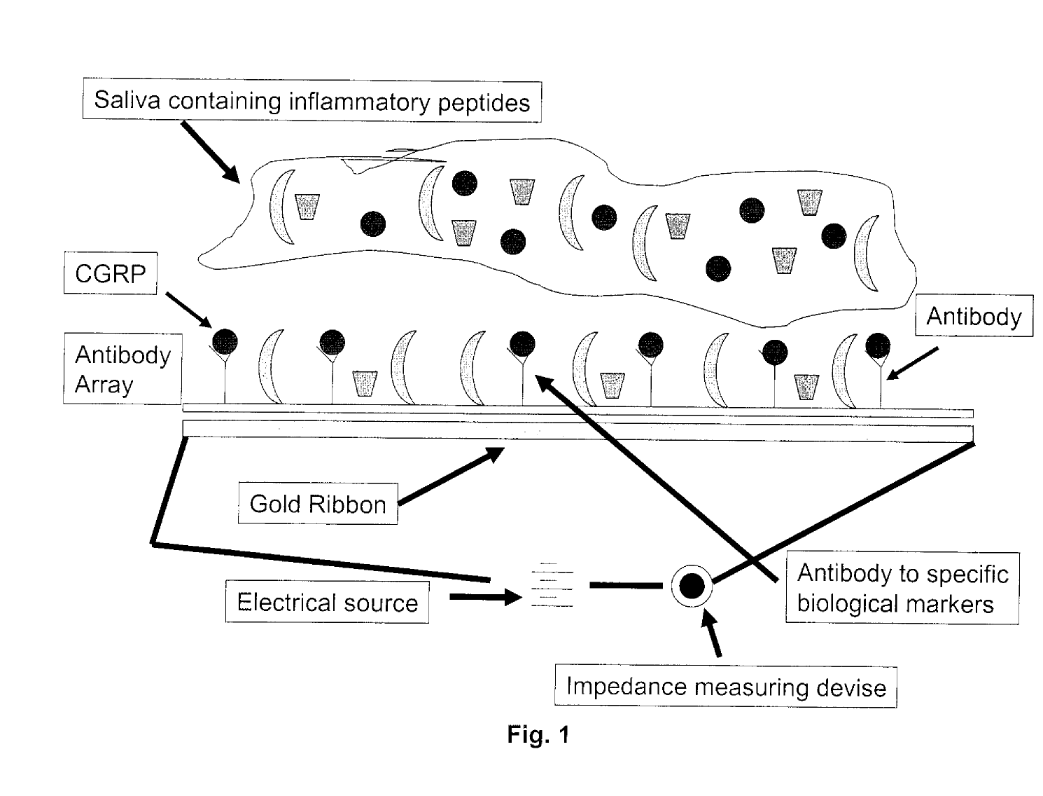

### DESCRIPTION OF THE PREFERRED EMBODIMENTS

In the following detailed description numerous specific details are set forth in order to provide a thorough understanding of the invention. However, it will be understood by those skilled in the art that the present invention may be practiced without these specific details. For example, the invention is not limited in scope to the particular type of industry application depicted in the figures. In other instances, well-known methods, procedures, and components have not been described in detail so as not to obscure the present invention.

Inflammatory peptides released during migraine, sinus headache, and rhinosinusitis can be measured in the salvia of an affected patient in a specific pattern that permits identification of the underlying pathophysiology. In addition, changes in inflammatory peptide patterns between attacks of episodic disease are uniquely different from those observed in patients with chronic disease patterns.

A medical device can be used to accurately measures changes of inflammatory peptides, proteins, human growth factors, endorphins, enkephalines, prostaglandins, and a variety of cytokines and other biological markers that are released as a result of underlying disease and pathophysiological change during primary and secondary headache disorders and other disease conditions. Specifically, saliva can be used as a diagnostic substrate containing biological markers for measuring these changes.

The described diagnostic method is advantageously well-suited to a known biosensor that has been developed. Application of the present method to such a biosensor permits measurement of changes in several biological markers in human saliva that occur with specific disease processes.

The method consists of providing a series of antibodies to several biological markers on a test strip in a known manner, including inflammatory peptides, proteins, human growth factors, endorphins, enkephalines, prostaglandins, and a variety of cytokines. A sample of saliva is collected from a human to be tested and applied to the series of antibodies. If the anticipated biological markers are present in the sample of saliva, those markers bind to their correlating antibodies thereby providing an indicator of the presence and amount of a particular biological marker in the patient's saliva upon evaluation of the test strip. A baseline sample may be taken and compared to subsequent samples in order to determine changes in the levels of specific biological markers in the patient over time.

When applied to a suitable biosensor, the described method can generate an electrical signal that can subsequently be transmitted to other devices. The biosensor consists of a substrate that is coated with D-poly lysine on which antibodies to various biological markers are randomly arranged. In preferred embodiment, the substrate comprises a gold ribbon. When saliva is applied to the biosensor, a biological marker present in the saliva will bind to its correlating antibody. The interaction of biological marker and antibody alters the electrical impedance of the substrate. This change in impedance can be readily measured and converted into a transmittable electrical signal. It is possible to use various salivatory proteins with this methodology to provide measurement and identification of biological markers critical in diagnosis, staging of disease progression, and treatment of many head and facial pain disorders.

The method described herein can identify, differentiate, diagnose, and stage the progression of disease for the purpose of directing appropriate treatment of diseases and disorders of headache and facial pain that involve the release of biological markers such as calcitonin-gene-related peptide, vasoactive intestinal peptide, neurokinin A and B, substance P, c-reactive protein, amylase, IgG, IgA, nitric oxide, prostaglandins, and histamine as a component of the disease process. The testing method can be applied to respiratory diseases, such as asthma and rhino sinusitis.

The preferred embodiments of the invention have been described above to explain the principles of the invention and its practical application to thereby enable others skilled in the art to utilize the invention in the best mode known to the inventors. However, as various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by the above-described exemplary embodiment, but should be defined only in accordance with the following claims appended hereto and their equivalents.

I claim:

1. A diagnostic test for head and facial pain in a human patient, comprising the steps of:
providing a test strip containing at least one antibody corresponding to at least one biological marker;
collecting a sample of saliva from said human patient;
applying said saliva sample to said test strip; and
evaluating said test strip for evidence of binding of said antibody with said biological marker present in said saliva sample.

2. The diagnostic test for head and facial pain as set forth in claim 1, wherein said biological marker is selected from the group consisting of inflammatory peptides, proteins, human growth factors, endorphins, enkephalins, prostaglandins, and cytokines.

3. The diagnostic test for head and facial pain as set forth in claim 1, wherein said biological marker is selected from the group consisting of calcitonin-gene-related peptide, vasoactive intestinal peptide, neurokinin A and B, substance P, c-reactive protein, amylase, IgG, IgA, nitric oxide, prostaglandins, and histamine.

4. The diagnostic test for head and facial pain as set forth in claim 1, wherein said step of providing a test strip further comprises providing said test strip in the form of a biosensor comprising a substrate having an electrical impedance and a coating, said at least one antibody being arranged on said coating.

5. The diagnostic test for head and facial pain as set forth in claim 4, wherein said coating comprises D-poly lysine.

6. The diagnostic test for head and facial pain as set forth in claim 4, wherein said substrate comprises a gold ribbon.

7. The diagnostic test for head and facial pain as set forth in claim 4, wherein said step of evaluating said biosensor further comprises measuring a change in said electrical impedance of said biosensor substrate.

8. The diagnostic test for head and facial pain as set forth in claim 7, further comprising the step of converting said change in said electrical impedance of said biosensor substrate into a transmittable electrical signal.

9. A diagnostic test for head and facial pain in a human patient, comprising the steps of
providing a biosensor comprising a substrate having an electrical impedance and a coating, at least one antibody corresponding to at least one biological marker on said coating;
collecting a sample of saliva from said human patient;
applying said saliva sample to said test strip; and
measuring a change in said electrical impedance of said biosensor substrate resulting from binding of said antibody with said biological marker present in said saliva sample.

10. The diagnostic test for head and facial pain as set forth in claim 9, further comprising the step of converting said change in said electrical impedance of said biosensor substrate into a transmittable electrical signal.

11. The diagnostic test for head and facial pain as set forth in claim 9, wherein said biological marker is selected from the group consisting of inflammatory peptides, proteins, human growth factors, endorphins, enkephalins, prostaglandins, and cytokines.

12. The diagnostic test for head and facial pain as set forth in claim 9, wherein said biological marker is selected from the group consisting of calcitonin-gene-related peptide, vasoactive intestinal peptide, neurokinin A and B, substance P, c-reactive protein, amylase, IgG, IgA, nitric oxide, prostaglandins, and histamine.

13. The diagnostic test for head and facial pain as set forth in claim 9, wherein said coating comprises D-poly lysine.

14. The diagnostic test for head and facial pain as set forth in claim 9, wherein said substrate comprises a gold ribbon.

15. A diagnostic test for head and facial pain in a human patient, comprising the steps of
providing a biosensor comprising a substrate having an electrical impedance and a coating, at least a first antibody and a second antibody corresponding to first and second biological markers being randomly arranged on said coating, said substrate comprising a gold ribbon and said coating comprising D-poly lysine;
collecting a sample of saliva from said human patient;
applying said saliva sample to said test strip;
measuring a change in said electrical impedance of said biosensor substrate resulting from binding of said antibody with said biological marker present in said saliva sample; and
converting said change in said electrical impedance of said biosensor substrate into a transmittable electrical signal.

16. The diagnostic test for head and facial pain as set forth in claim 15, wherein said biological marker is selected from the group consisting of inflammatory peptides, proteins, human growth factors, endorphins, enkephalins, prostaglandins, and cytokines.

17. The diagnostic test for head and facial pain as set forth in claim 15, wherein said biological marker is selected from the group consisting of calcitonin-gene-related peptide, vasoactive intestinal peptide, neurokinin A and B, substance P, c-reactive protein, amylase, IgG, IgA, nitric oxide, prostaglandins, and histamine.
